(19) United States
(12) Patent Application Publication
(10) Pub. No.: US 2026/0295284 A1
(43) Pub. Date: Oct. 1, 2026

(54) Electrode Assemblies for Tumor Treating Fields (TTFields) Therapy That Use a Dedicated Flex Circuit to Implement Temperature Sensing and Electrical Measurements

(71) Applicant: Novocure GmbH, Baar (CH)

(72) Inventors: Yoram WASSERMAN, Haifa (IL); Stas Obuchovsky, Haifa (IL); David Shapiro, Haifa (IL); Inbar Schlachet Drukerman, Haifa (IL)

(73) Assignee: Novocure GmbH, Baar (CH)

(21) Appl. No.: 19/634,430

(22) Filed: Mar. 31, 2026

Related U.S. Application Data
(60) Provisional application No. 63/781,158, filed on Mar. 31, 2025.

(51) Int. Cl. A61N 1/40

(52) U.S. Cl. CPC A61N 1/40

(57) ABSTRACT

Tumor treating Fields (TTFields) can be applied to a subject's body using an apparatus that comprises a flex circuit, at least one electrode element that is not integrated into the flex circuit, and one or more temperature sensors that are electrically connected to conductive traces of the flex circuit. A flexible backing is positioned behind and affixed to the electrode elements and the flex circuit. The electrode elements and the flex circuit are positioned so that, when viewed from a direction perpendicular to the flex circuit, the electrode elements lie within the outer boundary of the flex circuit.

### CROSS REFERENCE TO RELATED APPLICATIONS

This Application claims the benefit of U.S. Provisional Application 63/781,158, filed Mar. 31, 2025, which is incorporated herein by reference in its entirety.

### BACKGROUND

Tumor Treating Fields (TTFields) therapy is a proven approach for treating tumors using alternating electric fields at frequencies e.g., between 50 kHz-5 MHz, more commonly 100-500 KHz. Conventionally, the alternating electric fields are induced by electrode assemblies (e.g., arrays of capacitively coupled electrodes, also called transducer arrays) placed on the subject's skin on opposite sides of the subject's body. When an AC voltage is applied between opposing electrode assemblies, an AC current is coupled through the electrode assemblies and into the subject's body. And higher currents are strongly correlated with higher efficacy of treatment.

Alternating electric fields can also be used to treat medical conditions other than tumors. For example, as described in U.S. Pat. No. 10,967,167, alternating electric fields e.g., at 75-150 kHz can be used to increase the permeability of the blood brain barrier (BBB) so that, e.g., chemotherapy drugs can reach the brain.

One example of prior art electrode assemblies that can be used to apply alternating electric fields to a subject's body are the electrode assemblies described in U.S. Pat. No. 8,715,203. These electrode assemblies include nine electrode elements, each of which includes (a) a metal layer and (b) a ceramic layer with a very high dielectric constant positioned between the metal layer and the subject's skin. Thermistors positioned in small holes in the center of most of the electrode elements are included to make temperature measurements at those electrode elements.

Another example of prior art electrode assemblies that can be used to apply alternating electric fields to a subject's body are the electrode assemblies described in Pub. No. US 2021/0402179. These electrode assemblies have a flex circuit that includes a plurality of conductive pads on the front side of the flex circuit and a plurality of flexible polymer regions disposed over and in front of the conductive pads. A plurality of thermistors positioned on a rear side of the flex circuit in thermal contact with respective conductive pads are used to sense the temperature of the conductive pads.

To use either of these prior art electrode assemblies, an AC voltage is applied to the metal layers of the electrode elements in opposing electrode assemblies to generate the TTFields in the subject's body. The skin beneath the electrode elements heats up during use, resulting in a set of hotter spots immediately below the electrode elements, and a set of cooler regions immediately below the spaces between the electrode elements. And because safety considerations require that the skin temperature remains below a safety threshold (e.g., 41° C.), these hot spots limit the amount of current that can be delivered through the prior art electrode assemblies. More specifically, the prior art systems rely on signals from the thermistors to ensure that the current is low enough to prevent the temperature of the subject's skin from exceeding the safety threshold.

### SUMMARY OF THE INVENTION

One aspect of the invention is directed to a first apparatus for applying an electrical signal to a subject's body. The first apparatus comprises a flex circuit, at least one electrode element, one or more temperature sensors, and a flexible backing. The flex circuit has an insulating substrate and a plurality of conductive traces. The at least one electrode element is not integrated into the flex circuit and is also not integrated into a printed circuit board. Each of the one or more temperature sensors is electrically connected to at least one of the conductive traces of the flex circuit. The flexible backing is positioned behind the at least one electrode element and the flex circuit, and the at least one electrode element and the flex circuit are affixed to the flexible backing. The flex circuit has an outer boundary, and the at least one electrode element and the flex circuit are positioned so that, when viewed from a direction perpendicular to the flex circuit, the at least one electrode element lies within the outer boundary of the flex circuit.

In some embodiments of the first apparatus, the plurality of conductive traces of the flex circuit are formed using conductive ink. In some embodiments of the first apparatus, the at least one electrode element has a first area, the outer boundary of the flex circuit encompasses a second area, and the second area is larger than the first area.

Some embodiments of the first apparatus further comprise a first layer of conductive adhesive or conductive gel disposed on and in front of both the at least one electrode element and the flex circuit; a layer of graphite disposed on and in front of the first layer of conductive adhesive or conductive gel; and a second layer of conductive adhesive or conductive gel disposed on and in front of the layer of graphite.

Optionally, in the embodiments described in the previous paragraph, the at least one electrode element has a first area, the outer boundary of the flex circuit encompasses a second area, the second area is larger than the first area, and the first area is at least 10% of the second area. Optionally, in the embodiments described in the previous paragraph, the at least one electrode element has a first area, the outer boundary of the flex circuit encompasses a second area, the second area is larger than the first area, and the first area is at least 20% of the second area. Optionally, in the embodiments described in the previous paragraph, the at least one electrode element has a first area, the layer of graphite has a second area, and the first area is less than half of the second area. Optionally, in the embodiments described in the previous paragraph, the at least one electrode element has a first area, the layer of graphite has a second area, and the first area is less than one-quarter of the second area.

In some embodiments of the first apparatus, each of the electrode elements comprises a metal pad.

In some embodiments of the first apparatus, each of the electrode elements is or comprises a piece of metal foil. Optionally, these embodiments can further comprise a wire that is electrically connected directly to the piece of metal foil.

In some embodiments of the first apparatus, each of the electrode elements comprises a copper pad.

In some embodiments of the first apparatus, each of the electrode elements is or comprises a piece of copper foil. Optionally, these embodiments can further comprise a wire that is electrically connected directly to the piece of copper foil.

In some embodiments of the first apparatus, each of the electrode elements is or comprises a graphite sheet. Optionally, these embodiments can further comprise a wire that is electrically connected directly to the graphite sheet.

In some embodiments of the first apparatus, each of the electrode elements comprises a metal pad disposed on a rear surface of a layer of dielectric material having a dielectric constant of at least 10. In some embodiments of the first apparatus, each of the electrode elements comprises a layer of metal disposed on a rear surface of a ceramic plate having a dielectric constant of at least 1000.

Some embodiments of the first apparatus further comprise circuitry for implementing resistance, impedance, or conductance measurements between electrode elements on differing electrode assemblies positioned on the subject's body, and the circuitry is mounted to the flex circuit. Some embodiments of the first apparatus further comprise circuitry for implementing electrical impedance tomography, and the circuitry is mounted to the flex circuit. Some embodiments of the first apparatus further comprise circuitry for measuring evoked compound action potential, and the circuitry is mounted to the flex circuit.

Some embodiments of the first apparatus do not include a polyimide PCB or polyimide substrate.

Another aspect of the invention is directed to a second apparatus for applying an electrical signal to a subject's body. The second apparatus comprises a flex circuit, at least one electrode element, one or more temperature sensors, and a flexible backing. The flex circuit has an insulating substrate and a plurality of conductive traces. The at least one electrode element is not integrated into the flex circuit and is also not integrated into a printed circuit board. Each of the one or more temperature sensors is electrically connected to at least one of the conductive traces of the flex circuit. The flexible backing is positioned behind the at least one electrode element and the flex circuit, and the at least one electrode element and the flex circuit are affixed to the flexible backing. The at least one electrode element are positioned so that a convex hull that most closely circumscribes an outer boundary of the flex circuit lies outside a convex hull that most closely circumscribes the at least one electrode element.

In some embodiments of the second apparatus, the plurality of conductive traces are formed using conductive ink. In some embodiments of the second apparatus, the at least one electrode element has a first area, the outer boundary of the flex circuit encompasses a second area, and the second area is larger than the first area.

Some embodiments of the second apparatus further comprise a first layer of conductive adhesive or conductive gel disposed on and in front of both the at least one electrode element and the flex circuit; a layer of graphite disposed on and in front of the first layer of conductive adhesive or conductive gel; and a second layer of conductive adhesive or conductive gel disposed on and in front of the layer of graphite.

Optionally, in the embodiments described in the previous paragraph, the at least one electrode element has a first area, the outer boundary of the flex circuit encompasses a second area, the second area is larger than the first area, and the first area is at least 10% of the second area. Optionally, in the embodiments described in the previous paragraph, the at least one electrode element has a first area, the outer boundary of the flex circuit encompasses a second area, the second area is larger than the first area, and the first area is at least 20% of the second area. Optionally, in the embodiments described in the previous paragraph, the at least one electrode element has a first area, the layer of graphite has a second area, and the first area is less than half of the second area. Optionally, in the embodiments described in the previous paragraph, the at least one electrode element has a first area, the layer of graphite has a second area, and the first area is less than one-quarter of the second area.

In some embodiments of the second apparatus, each of the electrode elements comprises a metal pad.

In some embodiments of the second apparatus, each of the electrode elements is or comprises a piece of metal foil. Optionally, these embodiments can further comprise a wire that is electrically connected directly to the piece of metal foil.

In some embodiments of the second apparatus, each of the electrode elements comprises a copper pad.

In some embodiments of the second apparatus, each of the electrode elements is or comprises a piece of copper foil. Optionally, these embodiments can further comprise a wire that is electrically connected directly to the piece of copper foil.

In some embodiments of the second apparatus, each of the electrode elements is or comprises a graphite sheet. Optionally, these embodiments can further comprise a wire that is electrically connected directly to the graphite sheet.

In some embodiments of the second apparatus, each of the electrode elements comprises a metal pad disposed on a rear surface of a layer of dielectric material having a dielectric constant of at least 10. In some embodiments of the second apparatus, each of the electrode elements comprises a layer of metal disposed on a rear surface of a ceramic plate having a dielectric constant of at least 1000.

Some embodiments of the second apparatus further comprise circuitry for implementing resistance, impedance, or conductance measurements between electrode elements on differing electrode assemblies positioned on the subject's body, and the circuitry is mounted to the flex circuit. Some embodiments of the second apparatus further comprise circuitry for implementing electrical impedance tomography, and the circuitry is mounted to the flex circuit. Some embodiments of the second apparatus further comprise circuitry for measuring evoked compound action potential, and the circuitry is mounted to the flex circuit.

Some embodiments of the second apparatus do not include a polyimide PCB or polyimide substrate.

Another aspect of the invention is directed to a third apparatus for applying an electrical signal to a subject's body. The third apparatus comprises an AC voltage generator, at least two electrode assemblies, and at least two cables. The AC voltage generator generates at least one output signal at a frequency of 50 kHz-5 MHz. The at least two cables are arranged to route the at least one output signal to the at least two electrode assemblies. Each of the electrode assemblies includes (a) a flex circuit having an outer boundary, an insulating substrate, and a plurality of conductive traces, (b) at least one electrode element that is not integrated into the flex circuit and is also not integrated into a printed circuit board, (c) one or more temperature sensors, each of which is electrically connected to at least one of the conductive traces of the flex circuit, and (d) a flexible backing positioned behind the at least one electrode element and the flex circuit. The at least one electrode element and the flex circuit are affixed to the flexible backing, the flex circuit has an outer boundary, and the at least one electrode element and the flex circuit are positioned so that, when viewed from a direction perpendicular to the flex circuit, the at least one electrode element lies within the outer boundary of the flex circuit.

In some embodiments of the third apparatus, in each of the electrode assemblies, the plurality of conductive traces of the flex circuit are formed using conductive ink. In some embodiments of the third apparatus, in each of the electrode assemblies, the at least one electrode element has a first area, the outer boundary of the flex circuit has a second area, and the second area is larger than the first area.

In some embodiments of the third apparatus, each of the electrode assemblies further comprises (e) a first layer of conductive adhesive or conductive gel disposed on and in front of both the at least one electrode element and the flex circuit, (f) a layer of graphite disposed on and in front of the first layer of conductive adhesive or conductive gel, and (g) a second layer of conductive adhesive or conductive gel disposed on and in front of the layer of graphite.

In some embodiments of the third apparatus, each of the electrode elements comprises a metal pad. In some embodiments of the third apparatus, each of the electrode elements comprises a layer of metal disposed on a rear surface of a ceramic plate having a dielectric constant of at least 1000.

In some embodiments of the third apparatus, each of the electrode elements is or comprises a piece of metal foil. Optionally, these embodiments can further comprise a wire that is electrically connected directly to the piece of metal foil.

In some embodiments of the third apparatus, each of the electrode elements comprises a copper pad.

In some embodiments of the third apparatus, each of the electrode elements is or comprises a piece of copper foil. Optionally, these embodiments can further comprise a wire that is electrically connected directly to the piece of copper foil.

In some embodiments of the third apparatus, each of the electrode elements is or comprises a graphite sheet. Optionally, these embodiments can further comprise a wire that is electrically connected directly to the graphite sheet.

Some embodiments of the third apparatus further comprise circuitry for implementing resistance, impedance, or conductance measurements between electrode elements on differing electrode assemblies positioned on the subject's body, and the circuitry is mounted to the flex circuit. Some embodiments of the third apparatus further comprise circuitry for implementing electrical impedance tomography, and the circuitry is mounted to the flex circuit. Some embodiments of the third apparatus further comprise circuitry for measuring evoked compound action potential, and the circuitry is mounted to the flex circuit.

In some embodiments of the third apparatus, the at least one output signal has a frequency of 50-1000 kHz. In some embodiments of the third apparatus, the at least one output signal has a frequency of 100-500 kHz.

Another aspect of the invention is directed to a fourth apparatus for applying an electrical signal to a subject's body. The fourth apparatus comprises a flex circuit, at least one electrode element, one or more temperature sensors, and a flexible backing. The flex circuit has an insulating substrate and a plurality of conductive traces. The at least one electrode element is not integrated into the flex circuit. Each of the one or more temperature sensors is electrically connected to at least one of the conductive traces of the flex circuit.

The flexible backing is positioned behind the at least one electrode element and the flex circuit, and the at least one electrode element and the flex circuit are affixed to the flexible backing. The fourth apparatus also comprises (a) an evoked compound action potential measurement system that is mounted to the flex circuit and is electrically connected to at least one of the conductive traces of the flex circuit and/or (b) an electrical impedance tomography system that is mounted to the flex circuit and is electrically connected to at least one of the conductive traces of the flex circuit.

In some embodiments of the fourth apparatus, the flex circuit also has a plurality of conductive pads.

In some embodiments of the fourth apparatus, the flex circuit has an outer boundary. And the at least one electrode element and the flex circuit are positioned so that, when viewed from a direction perpendicular to the flex circuit, the at least one electrode element lies within the outer boundary of the flex circuit.

In some embodiments of the fourth apparatus, the at least one electrode element are positioned so that a convex hull that most closely circumscribes an outer boundary of the flex circuit lies outside a convex hull that most closely circumscribes the at least one electrode element.

In some embodiments of the fourth apparatus, the plurality of conductive traces of the flex circuit are formed using conductive ink.

Some embodiments of the fourth apparatus further comprise a first layer of conductive adhesive or conductive gel disposed on and in front of both the at least one electrode element and the flex circuit, a layer of graphite disposed on and in front of the first layer of conductive adhesive or conductive gel, and a second layer of conductive adhesive or conductive gel disposed on and in front of the layer of graphite.

### BRIEF DESCRIPTION OF THE DRAWINGS

FIG. 1 is a plan view of an apparatus for applying alternating electric fields (e.g., TTFields) to a subject's body.

FIG. 2 is a section view of the apparatus of FIG. 1.

FIG. 3 depicts an example of how to use the apparatus of FIG. 1 to apply alternating electric fields (e.g., TTFields) to a subject's body.

FIG. 4 is a plan view of another apparatus for applying alternating electric fields (e.g., TTFields) to a subject's body.

FIG. 5 is a section view of the apparatus of FIG. 4.

FIG. 6 depicts another apparatus for applying alternating electric fields (e.g., TTFields) to a subject's body.

FIG. 7 is a section view that depicts yet another apparatus for applying alternating electric fields (e.g., TTFields) to a subject's body.

FIG. 8 depicts an apparatus for applying alternating electric fields (e.g., TTFields) to a subject's body using a PCB and a flex circuit that overlap.

FIG. 9 depicts another apparatus for applying alternating electric fields (e.g., TTFields) to a subject's body using a PCB and a flex circuit that overlap.

FIGS. 10 and 11 are, respectively, plan and section views of another apparatus for applying TTFields to a subject's body that is similar to the FIG. 8 apparatus, but replaces the PCB with individual capacitively coupled electrode elements.

FIGS. 12 and 13 are, respectively, plan and section views of another apparatus for applying TTFields to a subject's body that is similar to the FIGS. 10-11 apparatus, but with a smaller flex circuit.

FIGS. 14 and 15 are, respectively, plan and section views of another apparatus for applying TTFields to a subject's body that is similar to the FIGS. 12-13 apparatus, but uses conductively coupled electrode elements.

FIG. 16 is a plan view of yet another apparatus for applying alternating electric fields (e.g., TTFields) to a subject's body.

Various embodiments are described in detail below with reference to the accompanying drawings, wherein like reference numerals represent like elements.

### DESCRIPTION OF THE PREFERRED EMBODIMENTS

Notably, in the two prior art designs described above, the hottest spots are always located directly beneath one of the electrode elements. It is therefore reasonable for these prior art systems to rely on temperature readings obtained at the electrode elements. But with other designs for electrode assemblies, obtaining temperature measurements only at the locations of the electrode elements may be insufficient to ensure that no portions of the electrode assembly heat up beyond the safety threshold. This can be particularly important in designs that use sheets of heat-conductive materials (e.g., graphite) to spread the heat out within any given electrode assembly. Examples of this type of electrode assembly are described in Pub. No. US 2023/0043071, which is incorporated herein by reference in its entirety.

Conventionally, TTFields therapy uses currents on the order of 1-6 Amps, and all functions of the electrode assembly are implemented using a single PCB that is robust enough to handle such currents without excessive heat generation. But high current PCBs are relatively inflexible, which can impact user comfort and compliance in wearing the electrode assemblies for extended periods. And the latter can negatively impact treatment effectiveness. High current PCBs are also relatively expensive to manufacture.

A different approach is to implement temperature sensing on a low current flex circuit that is separate from the PCB (or discrete electrode elements) that provides the relatively high current TTFields therapy. And when this approach is implemented, the resulting electrode assemblies can advantageously be made lighter, more flexible, and cheaper to manufacture. Furthermore, additional functionalities including but not limited to (a) evoked compound action potential (ECAP) measurements to assess electrosensation and (b) electrical impedance tomography (EIT) to explore electrode assembly positioning (and changes to electrode assembly positioning over extended treatment times) can optionally be implemented on the low current flex circuit.

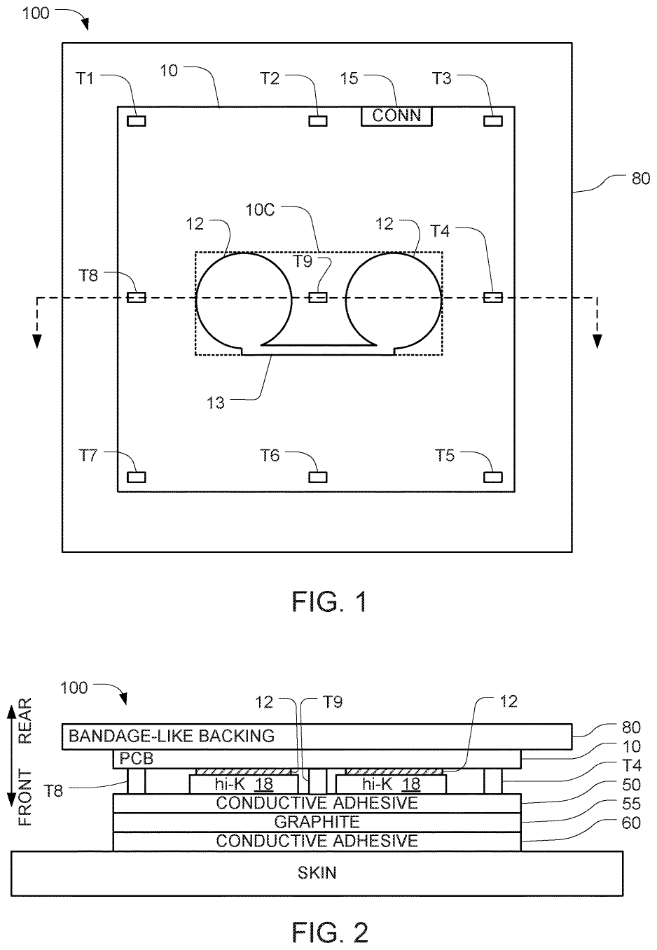

FIG. 1 is a plan view of an apparatus 100 (i.e., an electrode assembly) for applying alternating electric fields (e.g., TTFields) to a subject's body, and FIG. 2 is a section view of the same apparatus 100. The apparatus 100 includes at least one metal pad 12 positioned in the central portion of the apparatus, and a set of temperature sensors (e.g., thermistors) T1-T8 positioned at the peripheral portion of the apparatus is used to obtain temperature readings of the periphery of the apparatus.

The apparatus 100 uses a flexible PCB 10 that has a central section 10C and a peripheral section (i.e., the section that is located outside the outer boundary of the central section 10C). Note that as used herein, the term “PCB” refers to a printed circuit board, and this term encompasses rigid PCBs (e.g., with copper traces on a rigid epoxy board), as well as flexible PCBs (e.g., with copper traces on a flexible polyimide substrate). The term “flex circuit” refers to both flexible PCBs and printed circuits made by printing a conductive ink on a flexible substrate).

The PCB 10 has at least one metal pad 12 disposed on a front face of the central section 10C of the PCB, and a plurality of conductive traces (not shown). In use, the front face of the electrode assembly faces the skin of the subject. One or more temperature sensors T1-T8 (e.g., thermistors), or at least two temperature sensors, are mounted to the peripheral section of the PCB 10 (for example, four or more temperature sensors for electrode elements/metal pads distributed in a square or rectangular pattern). And these temperature sensors are electrically connected to at least one of the conductive traces. Optionally, one or more additional temperature sensors (e.g., thermistors) T9 may be positioned in the central portion of the apparatus 100. A connector 15 is mounted to the PCB 10, and this connector 15 is used to provide an electrical interface with the thermistors T1-T9 and the at least one metal pad 12. When more than one metal pad 12 is included (as depicted in FIGS. 1-2), all the metal pads 12 can be connected by conductive traces (e.g., metal traces) 13, in which case only a single pin of the connector 15 will be required to apply an AC signal to all of the metal pads 12. In alternative embodiments (not shown), the conductive traces 13 can be omitted, and each metal pad 12 can be electrically connected to its own individual pin on the connector 15.

In the embodiment depicted in FIGS. 1-2, the PCB 10 also includes a layer of dielectric material 18 disposed on and in front of the metal pads 12, and this layer of dielectric material 18 (labelled “hi-K” in FIG. 2) has a dielectric constant of at least 10. In some preferred embodiments, the layer of dielectric material 18 has a dielectric constant of at least 20 or at least 40. For example, the layer of dielectric material 18 may be a ceramic or a high dielectric constant polymer. A first layer of conductive adhesive 50 can be disposed in front of the PCB 10. In FIGS. 1-2, the first layer of conductive adhesive 50 is shown disposed on and in front of the PCB 10, and the rear surface of this conductive adhesive 50 adheres to the front surface of the dielectric material 18 and also to the front surfaces of the thermistors T1-T8 (and T9, if present). Note that in some embodiments, the layer of dielectric material 18 is not included, in which case the rear surface of the first layer of conductive adhesive 50 will adhere to the front surface of the metal pads 12 and also to the front surfaces of the thermistors T1-T8 (and T9, if present). Note also that in some embodiments, a layer of conductive gel (e.g., hydrogel) can be used in place of the first layer of conductive adhesive 50 depicted in FIG. 2.

A flexible backing 80 (e.g., a bandage-like backing) is positioned behind the PCB 10, and this flexible backing 80 is configured to support the PCB 10. At least a portion of the flexible backing 80 extends laterally beyond the PCB and the front of this portion is covered with a biocompatible adhesive that adheres to skin. This portion of the flexible backing 80 helps hold the apparatus 100 against the subject's skin.

The embodiment depicted in FIGS. 1-2 also includes a layer of anisotropic material (e.g., a sheet of graphite) 55 that is disposed on and in front of the first layer of conductive adhesive 50, and a second layer of conductive adhesive 60 disposed on and in front of the layer of anisotropic material 55. The layer of anisotropic material 55 can be a layer of pyrolytic graphite, graphitized polymer film, or graphite foil made from compressed high purity exfoliated mineral graphite. The layer of anisotropic material 55 has an area that is larger (e.g., at least twice as large) as an area of the central section 10C. The layer of anisotropic material 55 is preferably both thermally conductive and electrically conductive, and it acts to spread both the flow of current and heat in all four directions (i.e., to the right, to the left, into the page, and out of the page in FIG. 2, which corresponds to right, left, up, and down in FIG. 1). The second layer of conductive adhesive 60 should be biocompatible, and its function is to hold the apparatus 100 against the subject's skin. Note that in alternative embodiments, a layer of conductive gel (e.g., hydrogel) can be used in place of the second layer of conductive adhesive 60 depicted in FIG. 2.

In alternative embodiments, the layer of anisotropic material 55 and the second layer of conductive adhesive 60 can be omitted, in which case the first layer of conductive adhesive 50 should be biocompatible so that it can be adhered directly to the subject's skin.

Positioning the temperature sensors T1-T8 at the peripheral section of the apparatus 100 is particularly advantageous in embodiments that include the layer of anisotropic material 55, because unlike the prior art embodiments (in which it is virtually certain that the hottest spot will be directly beneath an electrode element), embodiments that include a layer of anisotropic material 55 spread out the current and heat over a much larger surface. And as a result, additional factors, including but not limited to geometric factors and anatomical factors, may impact which portion of the apparatus 100 will be the hottest. In one example, geometry comes into play because portions of opposing electrode assemblies that are closest to each other will tend to run hotter than the portions of opposing electrode assemblies that are spaced further apart. In another example, anatomy comes into play because sections of the apparatus 100 that overlie portions of the subject's skin with lower blood flow will tend to run hotter than sections that overlie portions of the subject's skin with higher blood flow (because blood flow carries heat away from the apparatus 100). Optionally, one or more additional temperature sensors T9 can be positioned at the central section 10C of the apparatus 100.

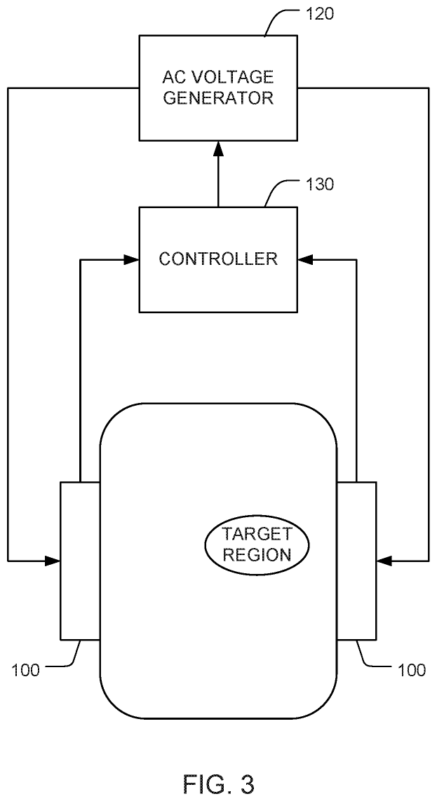

FIG. 3 depicts one example of how to use the apparatus 100 depicted in FIGS. 1-2 to apply alternating electric fields (e.g., TTFields) to a target region in a subject's body. Referring to FIGS. 1-3, a first apparatus 100 is adhered to the subject's skin on one side of the target region, and a second apparatus 100 is adhered to the subject's skin on the opposite side of the target region. To impose alternating electric fields in the target region, the AC voltage generator 120 applies an AC voltage with a frequency between 50 kHz and 5 MHz (e.g., 50-1000 kHz, 100-500 kHz, 75-300 kHz, or 150-250 kHz) between the metal pads 12 (FIGS. 1-2) of the first apparatus 100 and the metal pads 12 in the second apparatus 100. The AC signal from the AC voltage generator arrives at each of the first and second apparatuses 100 via a set of cables that terminate on the connector 15 of each apparatus 100. And from the connector 15, the signals are routed to the respective metal pads 12 via respective conductive traces (e.g., conductive metal traces, not shown) on the PCB 10.

The controller 130 ascertains the temperature of each of the temperature sensors T1-T8 (and T9, if present) in each of the first and second apparatuses 100 by inputting respective signals from those temperature sensors. Those signals arrive at the controller 130 via respective conductive traces on the respective PCB 10, respective connectors 15, and respective cables. If the controller 130 determines that all of the temperature sensors are below the threshold temperature, the controller 130 can increase the voltage of the AC signal, which will in turn increase the current that flows through the first and second apparatuses 100, which will in turn increase the intensity of the alternating electric field in the target region. On the other hand, if the controller 130 determines that any of the temperature sensors are at or approaching the threshold temperature, the controller 130 will decrease the voltage of the AC signal, which will decrease the current and eventually decrease the temperature of the hottest region of the apparatus 100.

Notably, the signals that the AC voltage generator 120 sends to the metal pads 12 of the first and second apparatuses 100 have relatively high currents (e.g., on the order of 1 A or even higher, for example 3-6 A). On the other hand, the signals that the controller 130 uses to ascertain the temperature of each of the temperature sensors T1-T8 (and T9, if present) is orders of magnitude lower (e.g., on the order of 1 mA or even lower). As a result, the PCB 10 can be implemented using relatively thick metal traces (e.g., copper traces) to route the signals from the connector 15 to the metal pads 12, and relatively thin metal traces (e.g., copper traces) to route the signals from the connector 15 to the temperature sensors T1-T8 (and T9, if present). And while this is not problematic from a technical perspective, it is sub-optimal from an economic perspective.

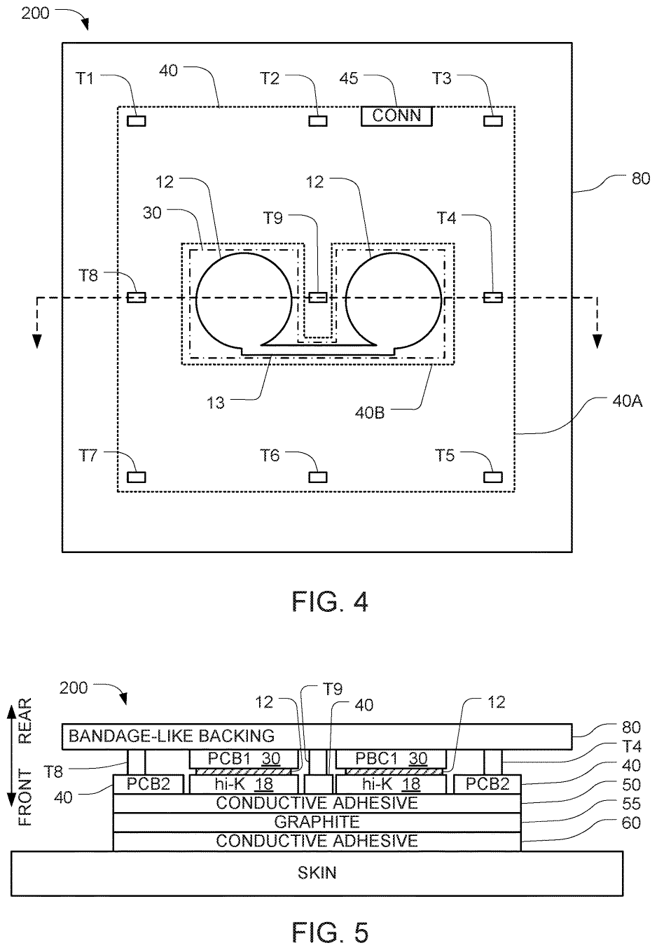

More specifically, because electrode assemblies have relatively large areas (e.g., on the order of 10-50 square inches), when a single PCB 10 is used to both route signals to the metal pads 12 (for applying TTFields) and route signals from the temperature sensors T1-T8 (and T9, if present) (for obtaining temperature readings), the single PCB will be relatively expensive. This is because when a single PCB 10 is used, the entire PCB must be designed to accommodate the highest expected current, even though only a relatively small portion of the PCB is actually used to carry higher currents. And large PCBs that can accommodate currents on the order of 1 A or higher are relatively expensive. The embodiments described below in connection with FIGS. 4-15 address this economic issue.

FIG. 4 is a plan view of another apparatus 200 (i.e., an electrode assembly) for applying alternating electric fields (e.g., TTFields) to a subject's body, and FIG. 5 is a section view of the same apparatus 200. The apparatus 200 includes a first PCB 30 positioned in the central portion of the apparatus, and a flex circuit 40 positioned at the peripheral portion of the apparatus. The first PCB 30 has an outer boundary (shown as a-.-line), and the first PCB and the flex circuit are positioned so that the flex circuit 40 lies outside the outer boundary of the first PCB 30.

The first PCB 30 in the central portion has a first substrate and at least one metal pad 12 disposed on the front face of the first substrate. When more than one metal pad 12 is included (as depicted in FIGS. 4-5), all the metal pads 12 can be connected by conductive traces (e.g., metal traces) 13.

Conductive metal traces (e.g., copper traces, not shown) on the first PCB 30 are used to route signals arriving from an AC voltage generator to the metal pads 12. A connector (not shown) may be included on the first PCB 30 to input these signals, or the signals can be electrically connected directly to one of the metal pads 12 or metal traces 13 using a hard-wired connection.

The flex circuit 40 in the peripheral portion of the apparatus 200 (having an outer boundary 40A and an inner boundary 40B, each shown as a dotted line in FIG. 4) is flexible, and it has a second insulating substrate with a plurality of conductive traces disposed thereon.

One or more, and preferably, at least two, temperature sensors T1-T8 (e.g., thermistors) are mounted to the flex circuit 40, and these temperature sensors are electrically connected to at least one of the conductive traces. Optionally, one or more additional temperature sensors T9 (e.g., thermistors) may be positioned in the central portion of the apparatus 200 (e.g., on the flex circuit 40, as depicted in FIG. 4). A connector 45 is mounted to the flex circuit 40, and this connector 45 is used to provide an electrical interface with the thermistors T1-T9.

In the embodiment depicted in FIGS. 4-5, the first PCB 30 also includes a layer of dielectric material 18 disposed on and in front of the metal pads 12, and this layer of dielectric material 18 (labelled “hi-K” in FIG. 5) has a dielectric constant of at least 10. In some preferred embodiments, the layer of dielectric material 18 has a dielectric constant of at least 20 or at least 40. For example, the layer of dielectric material 18 may be a ceramic or a high dielectric constant polymer.

A first layer of conductive adhesive 50 is disposed on and in front of both the first PCB 30 and the flex circuit 40, and the rear surface of this conductive adhesive 50 adheres to the front surface of the dielectric material 18 (of the first PCB 30). In some embodiments, the layer of dielectric material 18 is not included (e.g., as described below in connection with FIG. 7). Note that in alternative embodiments, a layer of conductive gel (e.g., hydrogel) can be used in place of the first layer of conductive adhesive 50 depicted in FIG. 5.

A flexible backing 80 (e.g., a bandage-like backing) is positioned behind both the first PCB 30 and the flex circuit 40, and this flexible backing 80 is configured to support both of those components 30, 40. Optionally, at least a portion of the flexible backing 80 extends laterally beyond the first PCB 30 and the flex circuit 40 and the front of this portion is covered with a biocompatible adhesive that adheres to skin. This portion of the flexible backing 80 helps hold the apparatus 200 against the subject's skin. Although the flexible backing 80 and the boundaries of the first PCB 30 and the flex circuit 40 are generally depicted herein for simplicity to be square or rectangular, they could, of course, be any shape including circular, oval, rounded triangular, etc.

The embodiment depicted in FIGS. 4-5 also includes a layer of anisotropic material (e.g., a sheet of graphite) 55 that is disposed on and in front of the first layer of conductive adhesive 50, and a second layer of conductive adhesive 60 disposed on and in front of the layer of anisotropic material 55. The layer of anisotropic material 55 has an area that is larger (e.g., at least twice as large) as the area of the first PCB 30. The layer of anisotropic material 55 is preferably both thermally conductive and electrically conductive, and it acts to spread both the flow of current and heat in all four directions as described above in connection with FIGS. 1-2. The second layer of conductive adhesive 60 should be biocompatible, and its function is to hold the apparatus 200 against the subject's skin. Note that in alternative embodiments, a layer of conductive gel (e.g., hydrogel) can be used in place of the second layer of conductive adhesive 60 depicted in FIG. 5.

In alternative embodiments, the layer of anisotropic material 55 and the second layer of conductive adhesive 60 can be omitted, in which case the first layer of conductive adhesive 50 should be biocompatible so that it can be adhered directly to the subject's skin.

Positioning the temperature sensors T1-T8 at the peripheral section of the apparatus 200 is advantageous for the same reasons described above in connection with the FIGS. 1-2 embodiment.

Notably, unlike the FIGS. 1-2 embodiment in which low current PCB traces (which are used to interface with the temperature sensors T1-T9) are implemented on the same PCB as high current traces (which are used to interface with the metal pads 12), all of the traces on the flex circuit 40 are low current traces. And this enables the flex circuit 40 to be made using very inexpensive technologies (e.g., with conductive traces made from conductive ink instead of copper). Moreover, because all of the high current traces are located on the first PCB 30, which is relatively small, the FIGS. 4-5 embodiment can be implemented using a relatively small high-current PCB (using expensive copper technology) and a relatively large low-current flex circuit that uses a less expensive (e.g., conductive ink) technology. And because the size of the expensive-technology PCB 30 in the FIGS. 4-5 embodiment is much smaller than the single, large, expensive-technology PCB 10 in the FIGS. 1-2 embodiment, the former can deliver a significant cost savings with respect to the latter.

The apparatus 200 depicted in FIGS. 4-5 is used in a manner that is similar to how the apparatus 100 (depicted in FIGS. 1-2) is used, as described above in connection with FIG. 3, with one notable exception. More specifically, instead of using a single connector 15 to route signals to and from both the thermistors T1-T8 (and T9, if present) and the metal pads 12 (as in the FIGS. 1-2 embodiment), the FIGS. 4-5 embodiment uses the connector 45 on the flex circuit 40 only to route signals to the thermistors T1-T8 (and, optionally, T9). And either an additional connector (not shown) or a hard-wired connection is used to route the AC TTFields signals to the metal pads 12.

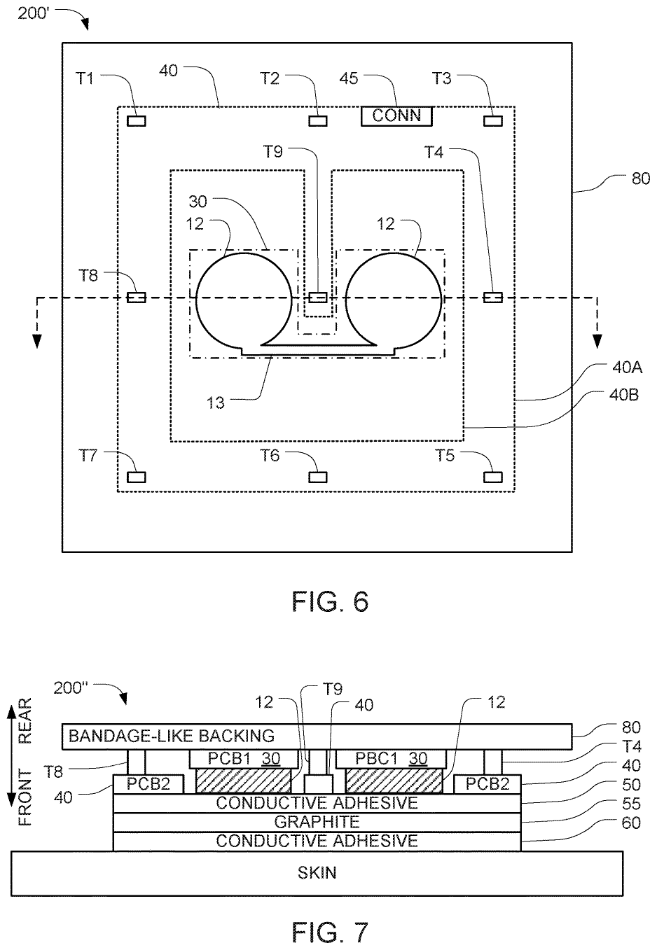

FIG. 6 depicts another apparatus 200′ that is similar to the apparatus 200 described above in connection with FIGS. 4-5, except that there is a larger space between the outer boundary of the first PCB 30 (shown as a-.-line) and the inner boundary 40B of the flex circuit 40 (shown as a dotted line). Use of this apparatus 200′ (and the labelling of the components of 200′ in FIG. 6) is similar to the use of the apparatus 200 (and the labelling of the components of 200 in FIG. 4-5) described above.

FIG. 7 depicts yet another apparatus 200″ that is also similar to the apparatus 200 described above in connection with FIGS. 4-5 (and with a similar labelling scheme), except that the layer of dielectric material 18 is omitted. As a result, the rear surface of the first layer of conductive adhesive 50 will adhere to the front surface of the metal pads 12 of the first PCB 30). This apparatus 200″ is used instead of the apparatus 200 described above in situations when it is desirable to conductively couple the AC signal from the AC voltage generator 120 (shown in FIG. 3) to the subject's body (as opposed to capacitively coupling that signal into the subject's body). Note that when the layer of dielectric material 18 is omitted, we avoid the problem of its interface resistance causing high heat. In addition, metal to metal contact with an area as small as 1 mm² can work at the current levels that are used for TTFields treatment. Use of this apparatus 200″ is similar to the use of the apparatus 200 described above. More specifically, a first apparatus 200″ is adhered to the subject's skin on one side of the target region, and a second apparatus 200″ is adhered to the subject's skin on the opposite side of the target region. And an AC voltage generator applies an AC voltage with a frequency between 50 kHz and 5 MHz (e.g., 50-1000 kHz, 100-500 kHz, 75-300 kHz, or 150-250 kHz) between the metal pads 12 of the first apparatus 200″ and the metal pads 12 in the second apparatus 200″.

In the embodiments described above in connection with FIGS. 4 and 6, there is a space between the outer boundary of the first PCB 30 (shown as a-.-line) and the inner boundary 40B of the flex circuit 40 (shown as a dotted line). This space is small in FIG. 4 and large in FIG. 6, and there is no portion of either FIG. 4 or 6 that shows the flex circuit 40 overlapping the first PCB 30. But in alternative embodiments, the flex circuit 40 can partially or completely overlap the first PCB 30.

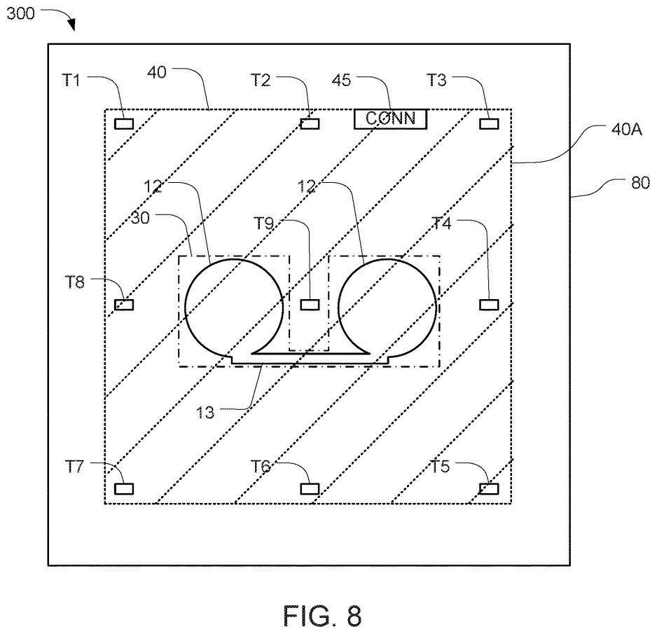

FIG. 8 is an example of such an embodiment 300. This FIG. 8 embodiment is similar to the FIG. 4 embodiment 200, except that the flex circuit 40 (drawn with diagonal crosshatching) is rectangular with no cut-out area. Preferably, at places where the first PCB 30 and the flex circuit 40 overlap, the flex circuit 40 is positioned behind the first PCB 30 (i.e., with the first PCB 30 in front and the flex circuit 40 in the rear.) In this example, the first PCB 30 has an outer boundary (shown as a-.-line), the flex circuit 40 has an outer boundary 40A (shown as a dotted line), and the first PCB 30 and the flex circuit 40 are positioned so that the outer boundary of the flex circuit 40 lies outside the outer boundary of the first PCB 30 (when viewed from a direction perpendicular to the first PCB 30). In FIG. 8, the flex circuit 40 completely overlaps the first PCB 30. Optionally, for embodiments where the flex circuit 40 partially or completely overlaps the first PCB 30, the first PCB 30 and the flex circuit 40 may be laminated together. Optionally, the laminate may comprise a conductive adhesive between the first PCB 30 and the flex circuit 40. Use of this apparatus 300 is similar to the use of the apparatus 200 described above. More specifically, a first apparatus 300 is adhered to the subject's skin on one side of the target region, and a second apparatus 300 is adhered to the subject's skin on the opposite side of the target region. And an AC voltage generator applies an AC voltage with a frequency between 50 kHz and 5 MHz (e.g., 50-1000 kHz, 100-500 kHz, 75-300 kHz, or 150-250 kHz) between the metal pads 12 of the first apparatus 300 and the metal pads 12 in the second apparatus 300.

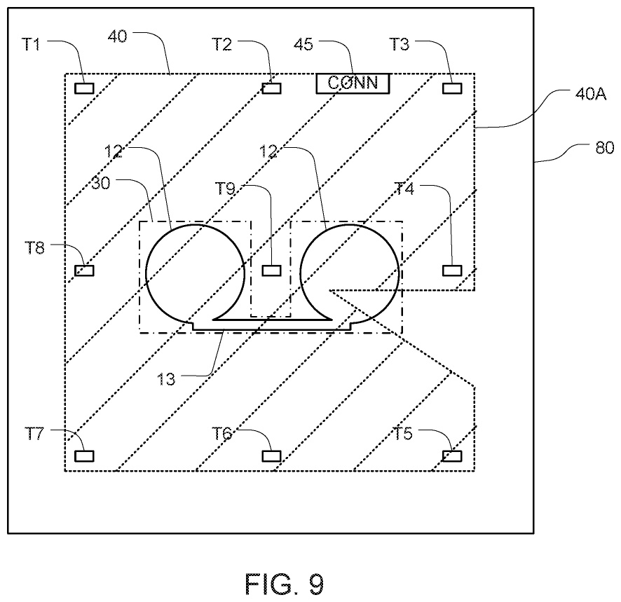

FIG. 9 is a variation of the FIG. 8 embodiment in which the flex circuit 40 has an irregular shape. In this situation, the first PCB 30 has an outer boundary (shown as a-. line), the flex circuit 40 has an outer boundary 40A (shown as a dotted line), and the first PCB 30 and the flex circuit 40 are positioned so that a rectangle that most closely circumscribes the outer boundary of the flex circuit 40 lies outside the outer boundary of the first PCB 30 (even though a portion of the first PCB 30 actually lies outside the outer boundary of the flex circuit 40).

The embodiments described above in connection with FIGS. 4-9 all employ a separate PCB 30 and flex circuit 40, with the higher current tasks (i.e., generating the electric field) being handled by the first PCB 30, and the lower current tasks (i.e., temperature sensing) being handled by the flex circuit 40. But in alternative embodiments, the higher current task of generating the electric field can be implemented using components or structures that are not mounted to any PCB or flex circuit. And in these embodiments, the first PCB 30 can be completely eliminated.

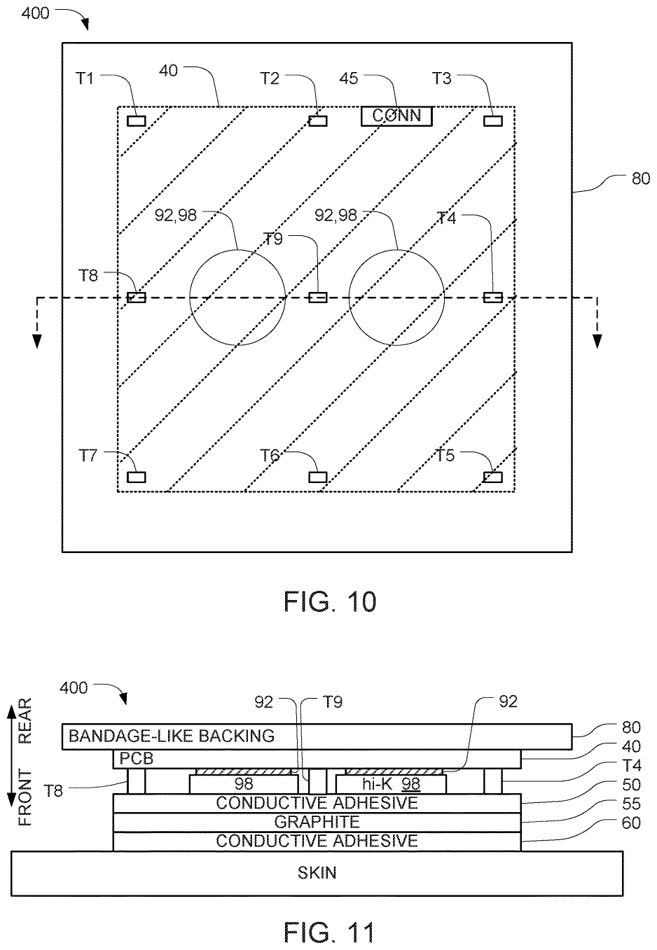

FIGS. 10 and 11 are, respectively, plan and section views of one example of such an embodiment 400. This embodiment 400 is generally similar to the FIG. 8 embodiment described above, except that the first PCB 30 and the metal pads 12 disposed on the first PCB 30 (in FIG. 8) are replaced by one or more ceramic plates 98 with a metallization layer 92 disposed on the rear surface of each of the ceramic plates 98 in FIGS. 10-11. Appropriate wiring (not shown) routes the AC signal that is used to generate the alternating electric fields to the metallization layer 92, and the ceramic plates 98 capacitively couple the current into the layer of conductive adhesive 50. The current will then flow forward through the layers 55 and 60, and will then flow into the subject's body. Note that to achieve efficient capacitive coupling of the AC signal, the dielectric constant of the ceramic plates 98 should be high (e.g., at least 1000, at least 2000, or at least 5000). In alternative embodiments, the ceramic plates 98 can be replaced with very thin layers of a polymer that have a dielectric constant of at least 10, which can also achieve efficient capacitive coupling. Note also that although the ceramic plates 98 and their metallization layers 92 are positioned in front of the flex circuit 40, they are not electrically connected to that flex circuit 40 and are not integrated into the flex circuit 40.

Because the ceramic plates 98 and their metallization layers 92 are not electrically connected to the flex circuit 40, there is no need for the flex circuit 40 to extend over that portion of the apparatus. Accordingly, in some embodiments, the size of the flex circuit can be reduced to the region that supports the temperature sensing components. Use of this apparatus 400 is similar to the use of the apparatus 100 described above in connection with FIG. 3. More specifically, a first apparatus 400 is adhered to the subject's skin on one side of the target region, and a second apparatus 400 is adhered to the subject's skin on the opposite side of the target region. And an AC voltage generator applies an AC voltage with a frequency between 50 kHz and 5 MHz (e.g., 50-1000 kHz, 100-500 kHz, 75-300 kHz, or 150-250 kHz) between the metallization layer 92 of the first apparatus 400 and the metallization layer 92 in the second apparatus 400.

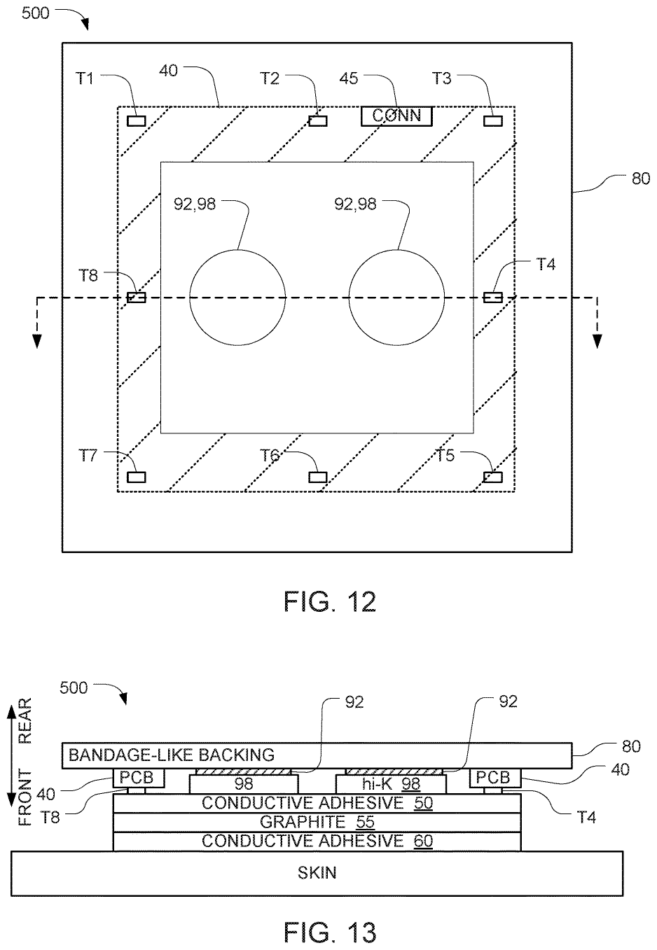

FIGS. 12 and 13 are, respectively, plan and section views of one example of such an embodiment 500. This embodiment 500 is generally similar to the FIGS. 10-11 embodiment 400 described above, except that the flex circuit 40 covers a smaller area. As described above in connection with the FIGS. 10-11 embodiment 400, the one or more ceramic plates 98 with a metallization layer 92 disposed on the rear surface of each of the ceramic plates 98 are used, and the ceramic plates 98 capacitively couple the current into the layers in front of the ceramic plates 98. And in this embodiment 500, the ceramic plates 98 and their metallization layers 92 are neither positioned in front of the flex circuit 40, nor are they electrically connected to that flex circuit 40. They are also not integrated into the flex circuit 40. Notably, in this embodiment, the ceramic plates 98 and their metallization layers are supported directly by the flexible backing 80. Use of this apparatus 500 is similar to the use of the apparatus 200 described above. More specifically, a first apparatus 500 is adhered to the subject's skin on one side of the target region, and a second apparatus 500 is adhered to the subject's skin on the opposite side of the target region. And an AC voltage generator applies an AC voltage with a frequency between 50 kHz and 5 MHz (e.g., 50-1000 kHz, 100-500 kHz, 75-300 kHz, or 150-250 kHz) between the metallization layer 92 of the first apparatus 500 and the metallization layer 92 in the second apparatus 500.

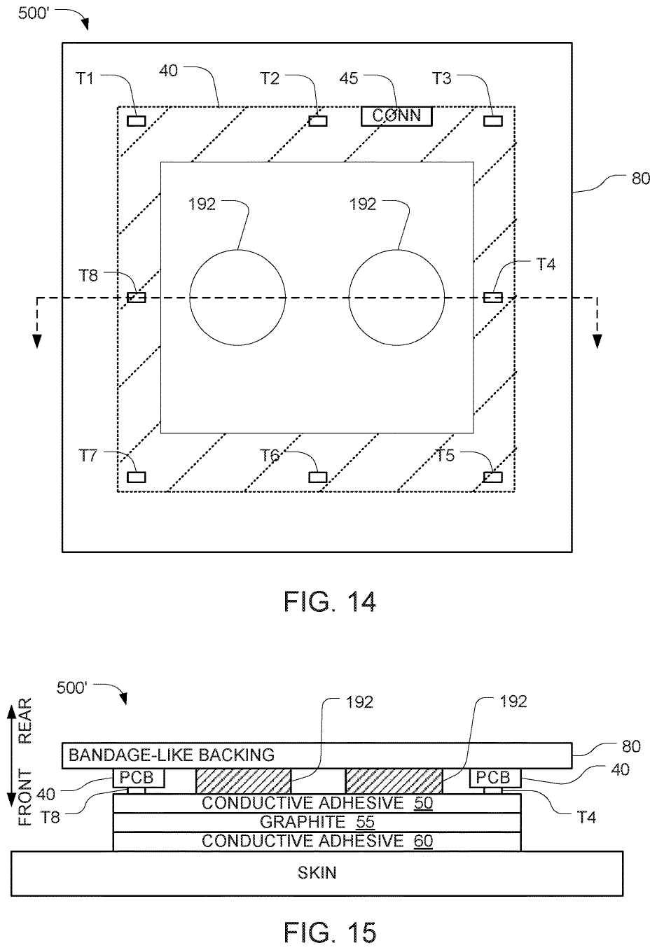

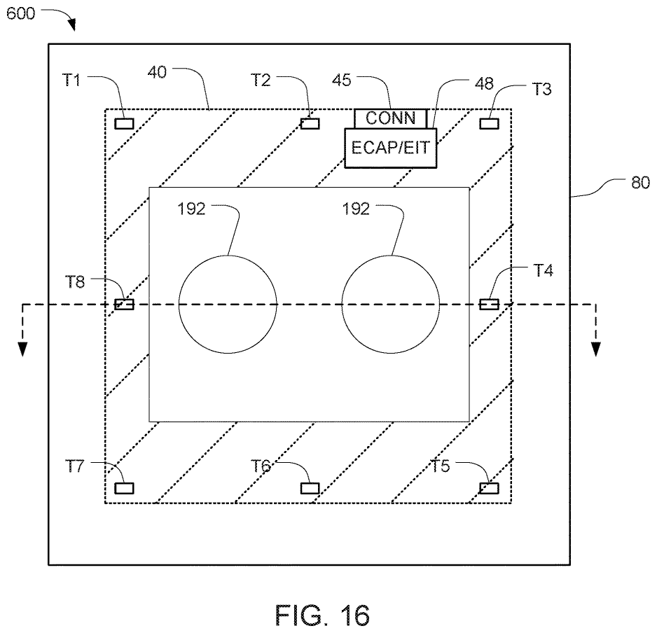

In a variation of this embodiment 500, the AC signal can be conductively coupled into the front layers 50, 55, and 60 instead of capacitively coupled into those layers. When conductive coupling is used, the ceramic plate 98 is omitted, and the signal can be applied to at least one piece of metal 192 (e.g., thin metal plates or metal foil) that are directly coupled to the layer of conductive adhesive 50 and are not integrated into the flex circuit 40, as shown in FIGS. 14-15. The remainder of the components in this FIGS. 14-15 embodiment 500′ are similar to the correspondingly numbered components in the FIGS. 12-13 embodiment 500. Use of this apparatus 500′ is similar to the use of the apparatus 200 described above. More specifically, a first apparatus 500′ is adhered to the subject's skin on one side of the target region, and a second apparatus 500′ is adhered to the subject's skin on the opposite side of the target region. And an AC voltage generator applies an AC voltage with a frequency between 50 kHz and 5 MHz (e.g., 50-1000 kHz, 100-500 kHz, 75-300 kHz, or 150-250 kHz) between the piece of metal 192 of the first apparatus 500′ and the piece of metal 192 in the second apparatus 500′.

Note that while FIG. 14 depicts two round pieces of metal 92, each of which serves as a respective electrode element, the electrode elements 92 can be configured in shapes that are not round (including but not limited to rectangular strips, various polygons, and irregularly shaped shapes).

Notably, in this FIGS. 14-15 embodiment, the electrode elements 92 are discreet elements that are not integrated into the flex circuit 40. Furthermore, the electrode elements 92 are not integrated into any printed circuit board whatsoever (which is unlike the situation described above in connection with FIGS. 4-7). Returning to FIGS. 14-15, the at least one electrode element 92 and the flex circuit 40 are positioned so that, when viewed from a direction perpendicular to the flex circuit 40, the at least one electrode element 92 lies within the outer boundary of the flex circuit 40.

In 2D geometry, a convex hull of a set of objects is the smallest convex polygon that encloses all of the objects. And notably, in this FIGS. 14-15 embodiment, all the electrode elements 92 are positioned so that a convex hull that most closely circumscribes an outer boundary of the flex circuit 40 (which will be a rectangle in the FIG. 14 view) lies outside a convex hull that most closely circumscribes all the electrode elements 92.

Collectively, all the electrode elements 92 have a first area, and the layer of graphite has a second area. The first area can be less than half of the second area, less than one-quarter of the second area, less than 40% of the second area, less than 30% of the second area, less than 20% of the second area, or less than 10% of the second area.

Optionally, in any of the embodiments described above in connection with FIGS. 4-15, the lower-current flex circuit 40 can be used to provide added functionality (e.g., by supporting additional circuitry, components, and/or additional terminals). FIG. 16 is a plan view of one example of such an embodiment 600. This embodiment 600 is generally similar to the FIGS. 14-15 embodiment 500′ described above, except that it incorporates additional circuitry 48 that is mounted to the lower-current flex circuit 40 in order to provide added functionality.

One example of adding circuitry 48 to the lower-current flex circuit 40 to provide added functionality is to mount an evoked compound action potential (ECAP) measurement system (e.g., based on an amplifier and an analog to digital converter and associated electrodes) to the lower-current flex circuit 40. The ECAP measurement system 48 is electrically connected to a plurality of conductive traces of the flex circuit 40. During certain types of electrical stimulation of biological tissue, the electrically evoked compound action potential represents the approximately synchronous firing of a population of electrically stimulated nerve fibers. Upon the application of an electrical signal of sufficient energy to activate nerve fibers, fibers of different diameters and in different locations are activated at roughly the same time (e.g., within fractions of milliseconds) and their action potentials (APs) propagate at different velocities to the vicinity of the ECAP electrodes.

Further, different nerve fibers of different diameters, which have different activation thresholds and conduction velocities, convey different signals, e.g., of types of sensation (vibration, temperatures, hair movement, muscle contraction-joint position, etc.).

ECAP signals can be measured using a set of electrodes that are positioned on a subject's skin and that connect to corresponding terminals on the lower-current flex circuit 40. These electrodes detect the compounded sum of the individual APs arriving at approximately the same time, which appear as a curve of a given amplitude and duration.

Signals from the ECAP measuring electrodes (which can be, e.g., on the order of m V) are forwarded to an ECAP measurement system. The ECAP measurement system processes those signals (e.g., using an amplifier and an analog to digital converter). These processed signals can subsequently be used e.g., to determine whether a subject is experiencing pain or electrosensation. Further details regarding ECAP and measurement thereof are provided, for example, in US Patent Application Publication No. 2023/0414955A1, entitled “Closed-Loop Technique to Reduce Electrosensation While Treating a Subject Using Alternating Electric Fields” and US Patent Application Publication No. 2024/0108887A1, entitled “Reducing Electrosensation While Treating a Subject Using Alternating Electric Fields by Deactivating Selected Electrode Elements,” each of which is incorporated herein by reference in its entirety. As described in the latter reference (US 2024/0108887 A1), with the appropriate circuitry, one can selectively deactivate one or more electrodes that are associated with experiencing pain or electrosensation.

The lower-current flex circuit 40 can also be utilized for resistance, conductance, or impedance measurements that could be used for current density estimations for optimizing the positioning layout of the electrode assemblies on parts of the subject's body (for example, by measuring impedance from one electrode element on a first electrode assembly to another electrode element on a different electrode assembly, and comparing similar results for other pairs of electrode elements). With an appropriate switching circuit, one can select different pairs of electrode elements on different electrode assemblies, as described below. One can obtain rough projections by comparing the impedance between select pairs of electrode assemblies, or one can provide a more detailed tomographic map of impedance in volume elements (voxels) through select regions of the body (i.e. Electrical Impedance Tomography, EIT).

Accordingly, another example of adding circuitry 48 to the lower-current flex circuit 40 to provide added functionality is to mount an electrical impedance tomography system to the lower-current flex circuit 40, together with a set of terminals that interface with a set of conductive pads that are positioned on the subject's body. The EIT system 48 is electrically connected to a plurality of conductive traces of the flex circuit 40. Electrical impedance tomography can be performed by sequentially applying signals (e.g., AC signals at a suitable frequency) between selected ones of the conductive pads (e.g., between a conductive pad on a first electrode assembly that is positioned on a first location of the subject's body, and a conductive pad on a second electrode assembly that is positioned on a second location of the subject's body), and measuring the impedance or conductance that is encountered by those signals. This can be implemented by adding N electrodes (where Nis an integer) and a N-to-1 selector to each of the electrode assemblies that are positioned on the subject's body. Each of the N electrodes can comprise a small pad on the lower-current flex circuit 40.

Thus, if four electrode assemblies are positioned at four different locations on the subject's body, there will be a total of 4×N electrodes on the subject's body. Each of these electrodes is connected to one of the N-to-1 selectors (i.e., with one N-to-1 selector positioned on each of the four electrode assemblies). The selectors can be connected to a set of input amplifiers and to a set of output amplifiers. The output amplifiers are configured to drive current, and the input amplifiers are configured to measure voltage. By controlling the selectors, we can access any given pair of electrodes, and we can use each pair of electrodes to sequentially measure a corresponding impedance. Conclusions derived from such electrical impedance tomography measurements can then be used e.g., to modify a course of the subject's treatment using TTFields. Further details regarding EIT and measurements made to create a tomographic map thereof are provided, for example, in US Patent Application Publication No. 2022/0313992 A1, entitled “Impedance Tomography Using Electrodes of a Tumor Treating Fields (TTFields) System,” which is incorporated herein by reference in its entirety. It should be noted that the system 600 depicted in FIG. 16 differs from that described in the reference because in the embodiments described herein the measurements are made from electrode elements (e.g. pads) on the low current flex circuit 40 instead of from the same electrode elements that are used to deliver TTFields to the subject. Use of this apparatus 600 is similar to the use of the apparatus 200 described above. More specifically, a first apparatus 600 is adhered to the subject's skin on one side of the target region, and a second apparatus 600 is adhered to the subject's skin on the opposite side of the target region. And an AC voltage generator applies an AC voltage with a frequency between 50 kHz and 5 MHz (e.g., 50-1000 kHz, 100-500 kHz, 75-300 kHz, or 150-250 kHz) between the piece of metal 192 of the first apparatus 600 and the piece of metal 192 in the second apparatus 600.

Headings are provided for convenience only and are not to be construed to limit the disclosure in any manner. Embodiments illustrated under any heading or in any portion of the disclosure may be combined with embodiments illustrated under the same or any other heading or other portion of the disclosure. Any combination of the elements described herein in all possible variations thereof is encompassed by the disclosure unless otherwise indicated herein or otherwise clearly contradicted by context. For example, and without limitation, embodiments described in dependent claim format for a given embodiment (e.g., the given embodiment described in independent claim format) may be combined with other embodiments (described in independent claim format or dependent claim format).

While the present invention has been disclosed with reference to certain embodiments, numerous modifications, alterations, and changes to the described embodiments are possible without departing from the sphere and scope of the present invention, as defined in the appended claims. Accordingly, it is intended that the present invention not be limited to the described embodiments, but that it has the full scope defined by the language of the following claims, and equivalents thereof.

What is claimed is:

1. An apparatus for applying an electrical signal to a subject's body, the apparatus comprising:
a flex circuit having an insulating substrate and a plurality of conductive traces;
at least one electrode element, wherein the at least one electrode element is not integrated into the flex circuit, and wherein the at least one electrode element is not integrated into a printed circuit board;
one or more temperature sensors, each of which is electrically connected to at least one of the conductive traces of the flex circuit; and
a flexible backing positioned behind the at least one electrode element and the flex circuit, wherein the at least one electrode element and the flex circuit are affixed to the flexible backing,
wherein the flex circuit has an outer boundary, and wherein the at least one electrode element and the flex circuit are positioned so that, when viewed from a direction perpendicular to the flex circuit, the at least one electrode element lies within the outer boundary of the flex circuit.

2. The apparatus of claim 1, wherein the plurality of conductive traces of the flex circuit are formed using conductive ink.

3. The apparatus of claim 1, further comprising:
a first layer of conductive adhesive or conductive gel disposed on and in front of both the at least one electrode element and the flex circuit;
a layer of graphite disposed on and in front of the first layer of conductive adhesive or conductive gel; and
a second layer of conductive adhesive or conductive gel disposed on and in front of the layer of graphite.

4. The apparatus of claim 3, wherein the at least one electrode element has a first area, the layer of graphite has a second area, and the first area is less than half of the second area.

5. The apparatus of claim 1, wherein each of the electrode elements is or comprises a piece of metal foil.

6. The apparatus of claim 5, further comprising a wire that is electrically connected directly to the piece of metal foil.

7. The apparatus of claim 1, wherein each of the electrode elements comprises a metal pad disposed on a rear surface of a layer of dielectric material having a dielectric constant of at least 10.

8. An apparatus for applying an electrical signal to a subject's body, the apparatus comprising:
a flex circuit having an insulating substrate and a plurality of conductive traces;
at least one electrode element, wherein the at least one electrode element is not integrated into the flex circuit, and wherein the at least one electrode element is not integrated into a printed circuit board;
one or more temperature sensors, each of which is electrically connected to at least one of the conductive traces of the flex circuit; and
a flexible backing positioned behind the at least one electrode element and the flex circuit, wherein the at least one electrode element and the flex circuit are affixed to the flexible backing,
wherein the at least one electrode element are positioned so that a convex hull that most closely circumscribes an outer boundary of the flex circuit lies outside a convex hull that most closely circumscribes the at least one electrode element.

9. The apparatus of claim 8, wherein the plurality of conductive traces are formed using conductive ink.

10. The apparatus of claim 8, further comprising:
a first layer of conductive adhesive or conductive gel disposed on and in front of both the at least one electrode element and the flex circuit;
a layer of graphite disposed on and in front of the first layer of conductive adhesive or conductive gel; and
a second layer of conductive adhesive or conductive gel disposed on and in front of the layer of graphite.

11. The apparatus of claim 10, wherein the at least one electrode element has a first area, the layer of graphite has a second area, and the first area is less than half of the second area.

12. The apparatus of claim 8, wherein each of the electrode elements is or comprises a piece of metal foil.

13. The apparatus of claim 12, further comprising a wire that is electrically connected directly to the piece of metal foil.

14. The apparatus of claim 8, wherein each of the electrode elements comprises a metal pad disposed on a rear surface of a layer of dielectric material having a dielectric constant of at least 10.

15. An apparatus for applying an electrical signal to a subject's body, the apparatus comprising:
a flex circuit having an insulating substrate and a plurality of conductive traces;
at least one electrode element, wherein the at least one electrode element is not integrated into the flex circuit;
one or more temperature sensors, each of which is electrically connected to at least one of the conductive traces of the flex circuit;
a flexible backing positioned behind the at least one electrode element and the flex circuit, wherein the at least one electrode element and the flex circuit are affixed to the flexible backing; and
at least one of (a) an evoked compound action potential measurement system that is mounted to the flex circuit and is electrically connected to at least one of the conductive traces of the flex circuit and (b) an electrical impedance tomography system that is mounted to the flex circuit and is electrically connected to at least one of the conductive traces of the flex circuit.

16. The apparatus of claim 15, wherein the flex circuit also has a plurality of conductive pads.

17. The apparatus of claim 15, wherein the flex circuit has an outer boundary, and wherein the at least one electrode element and the flex circuit are positioned so that, when viewed from a direction perpendicular to the flex circuit, the at least one electrode element lies within the outer boundary of the flex circuit.

18. The apparatus of claim 15, wherein the at least one electrode element are positioned so that a convex hull that most closely circumscribes an outer boundary of the flex circuit lies outside a convex hull that most closely circumscribes the at least one electrode element.

19. The apparatus of claim 15, wherein the plurality of conductive traces of the flex circuit are formed using conductive ink.

20. The apparatus of claim 15, further comprising:
a first layer of conductive adhesive or conductive gel disposed on and in front of both the at least one electrode element and the flex circuit;
a layer of graphite disposed on and in front of the first layer of conductive adhesive or conductive gel; and
a second layer of conductive adhesive or conductive gel disposed on and in front of the layer of graphite.
